(19) United States
(12) Patent Application Publication
(10) Pub. No.: US 2016/0183903 A1
(43) Pub. Date: Jun. 30, 2016

(54) INPUT DEVICE, METHOD, AND SYSTEM FOR GENERATING A CONTROL SIGNAL FOR A MEDICAL DEVICE

(71) Applicant: GENERAL ELECTRIC COMPANY, Schenectady, NY (US)

(72) Inventors: Stephane Vandroux, Paris (FR); Romain Moulin, Paris (FR); Renaud Heitz, Paris (FR)

(21) Appl. No.: 14/975,107

(22) Filed: Dec. 18, 2015

(30) Foreign Application Priority Data
Dec. 19, 2014 (GB) 1422720.1
Dec. 30, 2014 (GB) 1423358.9

(51) Int. Cl. A61B 6/00; A61B 34/00

(52) U.S. Cl. CPC A61B 6/547; A61B 34/25; A61B 6/563; A61B 2090/374

(57) ABSTRACT

Method and system for generating a control signal for operating a medical device using an input device located in a determined environment, wherein the input device comprises an input sensor for generating instructions for the medical device. The method comprises identifying the orientation or location of the input device in the determined environment generating a parameter linked to the identified location or orientation of the input device in the determined environment; using the identified orientation of the medical device or the identified location of the input device to identify a relative orientation of the input device with respect to the medical device; generating a parameter linked to said relative orientation of the input device with respect to the medical device; using the input device to generate an input signal relating to an instruction for the medical device; and using the input signal in combination with the parameter to obtain the control signal for the medical device.

### CROSS-REFERENCE TO RELATED APPLICATIONS

This patent application claims foreign priority benefits under 35 U.S.C. §119 to United Kingdom Patent Application No. 1422720.1, filed Dec. 19, 2014, and United Kingdom Patent Application No. 1423358.9 filed Dec. 30, 2014, which are hereby incorporated by reference in their entireties.

### BACKGROUND

The present disclosure relates to a method and an apparatus for generating a control signal for a medical device.

Medical devices, such as medical imaging devices, are used in combination with many other medical devices. These other devices are, for example, devices provided with a screen for monitoring data. Other devices include medical instruments like injectors or support devices such as tables. In order to operate the variety of devices an operator has to use a variety of input devices. Such input devices include remote controls, joysticks, push buttons, or similar input devices. The more devices used in an environment, the more difficult the operation of the different devices becomes. A related problem is the relative orientation of a medical device to the input device used to generate instructions for the medical device. If the relative orientation changes, the user can ideally continue to use the input device in an intuitive manner. Situations wherein the change in the relative orientation makes the intended use of the input device more complicated should be avoided. As a consequence, there is an apparent need for intuitive input devices which help operators to interact safely and easily with the medical devices they operate.

### SUMMARY OF THE INVENTION

In one aspect, the present disclosure is directed to a method for generating a control signal for operating a medical device using an input device located in a determined environment, wherein the input device is configured to generate instructions for the medical device, the method comprising:
identifying the location of the input device in the determined environment,
identifying the orientation of the medical device in the determined environment by an electronic compass or other orientation sensor or combination of sensors,
identifying the location and orientation of a medical device,
using the identified orientation of the medical device and the identified orientation of the input device to identify a relative orientation or location of the input device with respect to the medical device,
generating parameters linked to relative orientation or identified location of the input device,
using the input sensor of the input device to generate an input signal relating to an instruction for the medical device,
using the input signal in combination with the generated parameters to obtain a control signal for the medical device, or
using the control signal to operate the medical device. The method can be performed in any order, for example, the generation of an input signal using the input sensor can be done prior to the identification of the location or orientation of the input device in the determined environment.

In another aspect, the present disclosure is directed to a system for generating a control signal for operating a medical device in a determined environment, the system comprising:
a medical device configured to receive instructions by an input device,
an input device in communication with the medical device with an input sensor for generating an input signal relating to an instruction for the medical device,
the input device containing a sensor for identifying the orientation of the input device in the determined environment,
the input device containing a sensor, or other identifying indicia, capable of locating the input device in the determined environment,
placing at least one observation sensor in the determined environment to ascertain the locations of the input or medical devices in the determined environment,
wherein the at least one observation sensor is configured to generate a parameter relating the observed locations of the input or medical devices in the determined environment,
providing a processor or command and control system either in each device, or independent, configured to receive and process input and location signals from either the input device, the observation sensor, or both,
modulating a command and control signal for the medical device by accounting for the location, orientation, or both of the input device. As above, the method can be performed in any order with inputs from single sensors or combinations of sensors used to generate and transmit the control signal from the input device to the medical equipment.

In a further aspect, the present disclosure is directed to an input device for a medical device, the input device being configured for use in a determined environment, the input device having an input sensor for generating an input signal relating to an instruction for the medical device, wherein the input device is provided with an electronic compass, accelerometer, or other orientation sensor/sensor combination to identify the orientation of the input device in the determined environment.

At least one of the above aspects provides one or more solutions to the problems and disadvantages with the background art. Other technical advantages of the present disclosure will be readily apparent to one skilled in the art from the following description and claims. Various aspects of the present application obtain only a subset of the advantages set forth. No one advantage is critical to the aspects. Any described or claimed aspect or embodiment may be technically combined with any other claimed aspect(s) or embodiment(s).

### BRIEF DESCRIPTION OF THE DRAWINGS

The accompanying drawings illustrate example embodiments of the disclosure and serve to explain, by way of example, the principles of the disclosure.

FIG. 1 is a diagrammatic illustration of a determined environment, such as a hospital room, wherein an operator interacts with a variety of medical devices.

FIG. 2 is a diagrammatic illustration of a determined environment demonstrating a first location and orientation of an input device with respect to a medical device and a table for supporting a person.

FIG. 3 is a diagrammatic illustration of a second location and orientation of an input device with respect to a medical device and a table for supporting a person.

FIG. 4 is a diagrammatic illustration of an input device provided with a reference element.

FIG. 5 is a diagrammatic illustration of a bracelet with a reference element.

FIG. 6 shows the use of an input device in the form of a pointer device, for indicating a specific area of interest and axis of interest on an arm of a person.

FIG. 7 shows a flow chart of an embodiment of the method according to the present disclosure.

### DETAILED DESCRIPTION

In the present disclosure, the wording “determined environment” is used to indicate the environment where the medical device and the input device for the medical device are used. This “determined environment” can depend on the type of medical devices used. According to the present disclosure the “determined environment” makes reference to the fact that the medical device and the related input device are used in an environment which allows the identification of the location and/or the orientation of at least the input device. This location or orientation may be in reference either to the compositional elements of the determined environment itself (i.e., walls, ceiling, or floor of a room) or to equipment or markers within the determined environment. According to an embodiment of the invention it is possible that the locations and/or orientations of both the medical device and the input device can be determined either in reference to each other or derived from factors within the determined environment.

In the present disclosure the word “location” is used in relation to an input device and more particularly to the use of an input device in a determined environment. According to the present disclosure, the input device is configured to allow localization in three directions (X,Y,Z) and in three rotations to allow six degrees of freedom in a 3D environment. The word “location” refers to the position and/or the orientation of the input device in the determined environment.

In the present disclosure the phrase “input device” makes reference to a device which can be used to generate instruction for a medical device. The phrase “input device” makes reference to, for example, a joystick, an indicator in the form of a stick or pen, a wearable device with a location/orientation communicating aspect, a specific movement or gesture of the human body, or the mouse of a computer. The phrase “input device” refers to the fact that a movement of the input device can be translated into a specific instruction for the medical device to which the input device is connected.

In the present disclosure, the phrase “electronic compass” is used. The words “compass” and “electronic compass” make reference to a magnetometer and in particular a portable or mobile magnetometer or a device which provides a similar functionality.

In the present disclosure the phrase “medical device” is used to refer to a device which is typically configured to be used in a medical environment. The phrase “medical device” includes devices such as medical imaging devices, devices for monitoring data and other devices like injectors or support devices such as tables or beds.

In the present disclosure the abbreviation CAN is used. CAN makes reference to communication using a CAN bus. The letters CAN stand for “controller area network”. CAN bus is a message-based protocol and is specifically configured and used to allow microcontrollers and devices to communicate with each other without the use of a host computer.

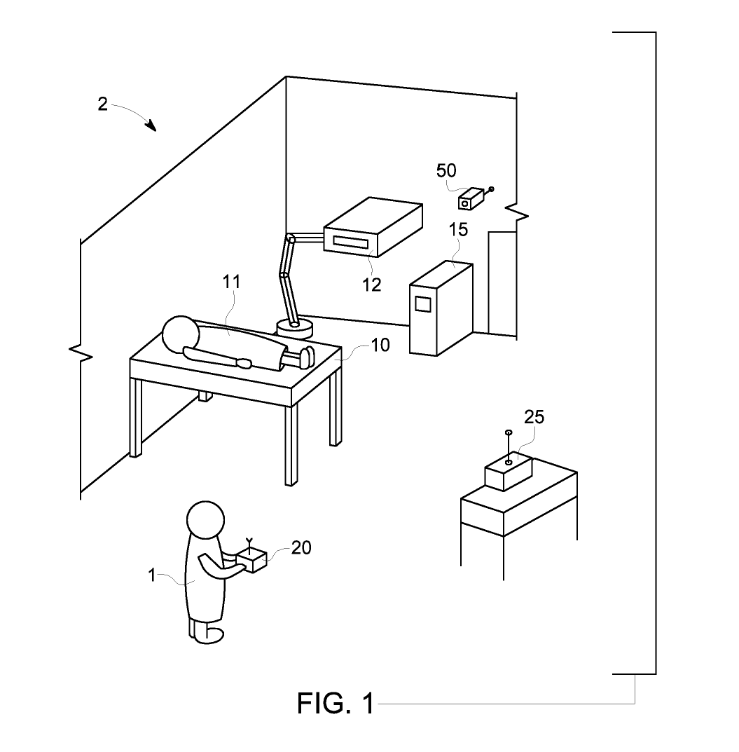

FIG. 1 is a diagrammatic illustration of a room 2 wherein a variety of medical devices are operated by an operator 1. The room 2 is for example a room in a medical institution, such as a hospital. The operator 1 is for example a medically skilled person such as a doctor or an assistant.

In the room 2 a first device 10 is present which is used as a table or support for supporting a person 11. This person 11 could be a patient of which physiological parameters of any type need to be obtained. In the room 2 a first medical device 12 is present for obtaining physiological parameters of a certain type. In order to operate the medical device 12 the operator 1 uses an input device 20. This input device 20 is, for example, a joystick which is connected to a control unit which is part of the medical device 12 or connected to the medical device 12, and which allows for controlling the movement of the medical device 12 with respect to the support 10. The input device 20 can be a wireless input device which can communicate, using a wireless communication protocol, with a medical device 12 or a control device connected to the medical device 12.

In the room 2 according to FIG. 1 a further medical device 15 is present which can be operated using a further input device 25. The input device 25 could be similar to the input device 20 or could be of a different type. It will be understood that the larger the number of medical devices to be operated by the operator 1, the more complicated the operation of the different devices becomes, especially if the operator 1 only uses the different medical devices 12, 15 occasionally.

An instruction provided by the input device 20 can be used to operate the medical device 12. A first instruction could, for example, relate to a required movement of the medical device 12 with respect to the person 11 in order to position the medical device 12 correctly with respect to said person 11. A second instruction could include a specific action to be executed by the medical device 12 in order to measure physiological parameters of the person 11.

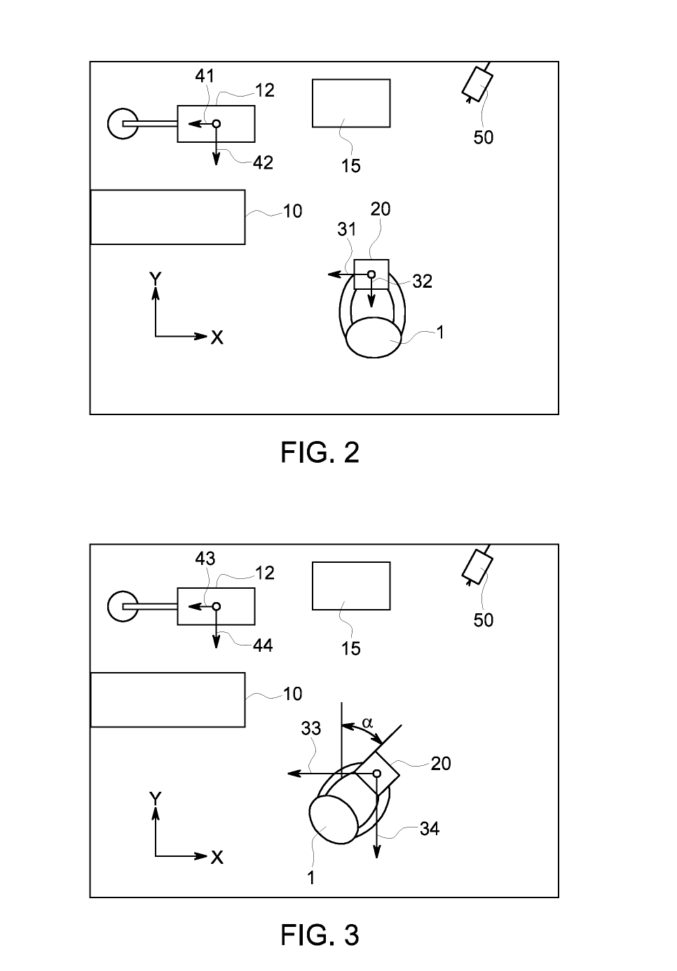

In the embodiment according to FIG. 1, the operator 1 uses a joystick 20 to move the medical device 12 to the required position. Preferably, the instruction provided by the joystick 20 will be followed in a logical and intuitive manner by the medical device 12. That means that if the operator moves the joystick in a certain direction, the medical device will preferably follow the generated instruction by moving in a manner similar to the movements of the joystick. With reference to FIG. 2 and FIG. 3, as an example, a specific use of the joystick for operating the medical device 12 is described.

FIG. 2 schematically shows room 2 in top view. FIG. 2 also shows in top view the input device 20 which has the form of a joystick. If the operator 1 would like to move the medical device 12 with respect to the support 10 the joystick could be used to provide instructions for the medical device 12 to move. A joystick movement in a direction indicated with arrow 31 could allow the medical device 12 to move in the direction 41 from right to left as seen in FIG. 2. A movement of the joystick in a direction indicated with arrow 32 could allow the medical device 12 to move in a direction 42 from the top towards the bottom according to FIG. 2. If no specific measures were taken, to allow the operator 1 to move the medical device 12 in an intuitive manner the operator 1 should be positioned correctly with respect to the medical device 12 to ensure that movement of the joystick in, for instance, direction 31, will be followed by a movement of the medical device 12 in a parallel direction 41.

Turning to FIG. 3, room 2 is represented in a further top view whereby the input device 20 has moved its position with respect to the position indicated in FIG. 2. A reason for this movement could be that the input device 20 is connected to the operator 1 and that the operator 1 has moved in the room 2. The result is that the input device 20 has rotated over an angle alpha which means that the input device 20 is no longer positioned in line with the medical device 12. If no specific measures were taken, the operator 1, in order to be able to use the joystick 20 in an intuitive manner, should first return to his position as indicated in FIG. 2 or a position parallel to that position to allow the movement of the joystick 20 to be followed by a movement of the medical device 12 in a direction parallel to the movement of the joystick 20.

In order to allow the operator 1 to continue using the input device 20 in an intuitive manner irrespective of the position of the operator 1 or the input device 20, according to the present disclosure room 2 is also provided with at least one observer sensor 50. The observer sensor 50 is configured on its own or in combination with further observer sensors 50 to determine the location, that is to say the position and/or the rotation of the input device 20 with respect to said at least one observer sensor 50. According to an embodiment of the disclosure the at least one observer sensor 50 is configured to determine the position and/or orientation of the medical device 12 with respect to a reference in the room, which allows the system to determine the relative location of the input device 20 with respect to the medical device 12. Returning to FIG. 3, if the operator 1 would like that movement of the joystick in the direction indicated with the arrow 33 is followed by movement of the medical device in a direction 43, the operator 1 does not need to compensate for any rotation or displacement of either the operator 1 or the input device 20 with respect to the medical device 12. The compensation would be automatic and the movement of the joystick in a direction of the arrow 33 being followed by a movement of the medical device in the direction 43 allows for intuitive use of the joystick 20.

According to the present disclosure, intuitive use is accomplished by the fact that an instruction for the medical device 12 generated by the input device 20 comprises two elements. The first element of the instruction for the medical device 12 will be generated by the input device 20 wherein, in the example of FIGS. 2 and 3, a movement of a joystick will generate an input signal relating to an instruction for the medical device 12 to move from a first position towards a second position. The second element of the instruction is dependent on the position and orientation of the input device and is provided by the at least one observer sensor 50. The sensor 50 will determine the location of the input device and will generate a parameter linked to the identified location of the input device 20. The actual control signal to operate the medical device 12 will comprise the input signal created by the input device 20 and the parameter obtained by the at least one observer sensor 50 which will compensate for any change in location of the input device 20. The effect is that the input signal generated by the input device 20 is automatically compensated for any displacement of the input device to continue to allow intuitive use of that input device 20.

According to an embodiment of the disclosure the parameter linked to the identified location of the input device 20 generated by the at least one observation sensor will be generated prior to, while or after an input signal has been generated by the input device 20. That means that the parameter will be generated for the specific location at which the input signal was generated by the input device 20.

According to an alternative embodiment of the disclosure the at least one observation sensor 50 could continuously generate a parameter linked to the location of the input device 20 and combine the so obtained parameter linked to the identified location of the input device 20 to a generated input signal, once such an input signal is generated by the input device 20.

In another embodiment, the input device 20, according to the present disclosure, is provided with an electronic compass. This electronic compass is configured to determine the orientation of the input device 20 in the room 2. It is possible to use a reference in the room 2 and to allow the electronic compass to determine its orientation with respect to said reference. According to an embodiment of the disclosure, the electronic compass is configured to determine the orientation of input device 20 with respect to a medical device 10, 12 or 15 in the room 2. The fact that the electronic compass is configured to determine the orientation of input device 20, means the system is able to determine the relative position of the input device 20 with respect to a reference position.

According to an embodiment of the disclosure in a first step, the orientation of the medical device 12, operated by the input device 20, is determined. In a further step the orientation of the input device 20 is obtained by the electronic compass. It is possible to calibrate the electronic compass using the initial position of the medical device 12. This would mean that the orientation of the input device 20 obtained by the electronic compass would relate to a relative orientation of the input device 20 with respect to the orientation of the medical device 12. As an alternative, it is possible that the medical device 12 would be provided with a second electronic compass. In that case, both the orientation of the medical device 12 and the input 20 are obtained by an electronic compass.

Once the orientation of both the medical device 12 and the input device 20 is known, a relative orientation of the input device with respect to the medical device 12 can be obtained. The relative orientation of the input device 20 with respect to the medical device 12 can be used to correct any instructions generated by the input device 20.

If the operator 1 would like movement of the joystick 20 in the direction indicated with arrow 33 to be followed by movement of the medical device 12 in a direction 43, the operator 1 does not need to compensate for any orientation, rotation on or displacement of either the operator 1 or the input device 20 with respect to the medical device 12. The correction of any instruction, with the objective of obtaining intuitive use of the input device 20, would be automatic and the movement of the joystick 20 in a direction of arrow 33 would be followed by a movement of the medical device 12 in the direction 43.

According to the present disclosure, intuitive use of the input device 20 is accomplished by the fact that an instruction for the second medical device 12 comprises two elements. The first element of the instruction for the medical device 12 will be generated by the input device 20 wherein, in the example of FIGS. 2 and 3, a movement of a joystick will generate an input signal relating to an instruction for the medical device 12 to move from a first towards a second position. The second element of the instruction is dependent on the relative orientation of the input device 20 with respect to the medical device 12. After the relative orientation between the input device 20 and the medical device 12 is obtained, a parameter will be generated which is linked to said identified relative orientation. The actual control signal to operate the medical device 12 will comprise the input signal created by the input device 20 and said parameter relating the relative orientation. The technical effect of the measures is that the input signal generated by the input device 20 is automatically compensated for any orientation or displacement of the input device 20, to continue to allow intuitive use of that input device 20.

According to an embodiment of the disclosure the electronic compass of the input device 20 could continuously generate a parameter linked to the relative orientation of the input device 20 with respect to the medical device 12. Moreover, it would be possible to identify an updated orientation of the medical device 12 in the determined environment. If the medical device 12 comprises an electronic compass, the orientation of the medical device 12 may be obtained continuously by said electronic compass.

In order to be able to able to execute the method according to the disclosure and to obtain the functionality described with respect to the drawings, a system for generating a control signal for operating a medical device 12 in a determined environment 2, according to the disclosure, comprises at least a medical device 12 configured to receive instructions generated by an input device 20. The system further comprises an input device 20 connected to the medical device 12. The connection could be obtained via a wired and/or a wireless connection. In the case of a wireless connection the input device 20 and the medical device 12 should be configured to communicate via an adapted wireless communication protocol. According to the disclosure the input device 20 is provided with an input sensor for generating an input signal relating to an instruction for the medical device. The input sensor is able to translate an instruction provided by the operator 1 into an input signal. The input device 20 further comprises an electronic compass for identifying the orientation of the input device 20 in the determined environment 2 wherein the input device 20 is used. The system further comprises a processor connected to the input device 20, wherein the processer is adapted to receive and process data relating to the orientation of the medical device 20 in the determined environment 2, data relating to the orientation of the input device 12 in the determined environment 2 obtained by the electronic compass, and to obtain an input signal generated by the input sensor. The processor could be physically present in the input device 20 or in the medical device 12 or could be present in a separate device connected to both the input device 20 and the medical device 12. The processor should be configured to process data, the data relating to the orientation of the medical device 20 in the determined environment 2, the data relating to the orientation of the input device 12 in the determined environment 2 obtained by the electronic compass, and an input signal generated by the input sensor to obtain, as a result of said processing, the control signal for the medical device.

In the system according to the disclosure the different devices 12 and 20 could communicate by CAN protocol or any other protocols sufficient to convey the relevant information.

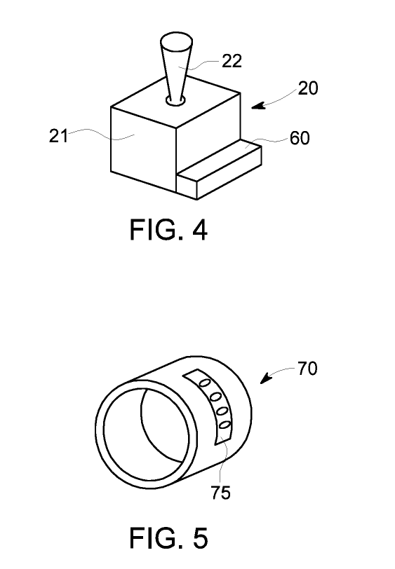

FIG. 4 shows schematically an input device 20 in the form of a joystick provided with a base element 21 and an input sender 22 configured to move relative to the base element 21 to thereby generate an input signal relating to an instruction for the medical device to which the input device 20 is connected. In order to allow the system described above with reference to FIGS. 1, 2 and 3 to determine the location of the input device 20, the input device 20 comprises a reference element 60. The element 60 is specifically configured to allow the at least one observer sensor 50 to recognize the element 60 and to allow the at least one observer sensor 50 to determine the location of the element 60 and thereby of the input device 20 to which the reference element 60 is attached.

In a first embodiment of the invention, the reference element 60 could comprise a passive element, for example in the form of an elongated or triangular member, which facilitates recognition of the position and/or orientation of the reference element 60. Such a passive element could for instance facilitate image recognition when, for example, the at least one observation sensor 50 comprises cameras that enable the identification of the location by providing, for example, 3D visualization.

The reference element 60 could also comprise, for example, an active element in the form of an LED element to facilitate recognition of the location of the reference element 60.

According to a further embodiment, the reference element comprises an element for allowing electromagnetism localization.

According to a further embodiment of the present disclosure, the reference element 60 is configured to allow radio-frequency (RF) localization such as RFID localization.

In FIG. 5 a bracelet 70 is shown schematically. In one embodiment, the bracelet 70 comprises a reference element 75 which has a similar function as the reference element 60 as described with reference to FIG. 4.

In at least one embodiment, the bracelet 70 could be worn by the operator 1 to allow the system as described with reference to FIGS. 1-3 to determine the location of the bracelet and thereby the location of the body part to which the bracelet 70 is attached. The possibility of locating a bracelet 70 in a determined environment such as the room 2 according to FIGS. 1, 2 and 3 will allow the operator 1 to interact with different medical devices in a determined environment while improving safety. If the plurality of medical devices is present in a determined environment, such as the room 2 according to FIG. 1 of the present disclosure, it is possible to enable operation of at least one of the plurality of medical devices such that the at least one medical device only receives instructions if the distance between the bracelet and the at least one medical device, or the distance between the bracelet and an input device for the at least one medical device, is equal to or below a certain set threshold value.

This means that if the input device comprises a touch screen, any instructions generated by touching the screen will only be taken into account if, at the same time, a determined distance between the bracelet 70 and the touch screen is equal to or below the threshold value. In at least one embodiment the threshold value is, for example, between about 10 centimeters and about 100 centimeters. And in another embodiment, the threshold value is about 20 centimeters. If the distance between the bracelet 70 and the touch screen exceeds the threshold value, then this implies that the touch screen was touched by coincidence and the operator who is supposed to be providing instructions via the touch screen is not in the direct vicinity of the touch screen. In other words, generating instructions is not possible by simply touching an input device unless the system has also confirmed that the operator who is supposed to be operating a certain device is effectively either close to the medical device or close to the input device associated with the medical device. The use of the bracelet 70 therefore increases the safety of operating one or more medical devices in a determined environment.

A further use of a bracelet 70 according to FIG. 5 is that an input signal can be generated by moving the bracelet 70 with respect to either a medical device or an input device. Both the movement and the location of the bracelet 70 can be used to generate an input signal. For example, in case of an emergency, if the operator touches the medical device several times to try and stop said device, the system according to the present disclosure can recognize the movement of the bracelet 70 and thus the movement of a body part to which the bracelet is attached to thereby recognizing an instruction to stop the operation of a medical device and/or any operating function of the medical device. Likewise the system according to the present disclosure can recognize the movement of the bracelet 70 and thus the movement of a body part to which the bracelet is attached to recognize an instruction to activate or commence operation of a medical device and/or any function of the medical device.

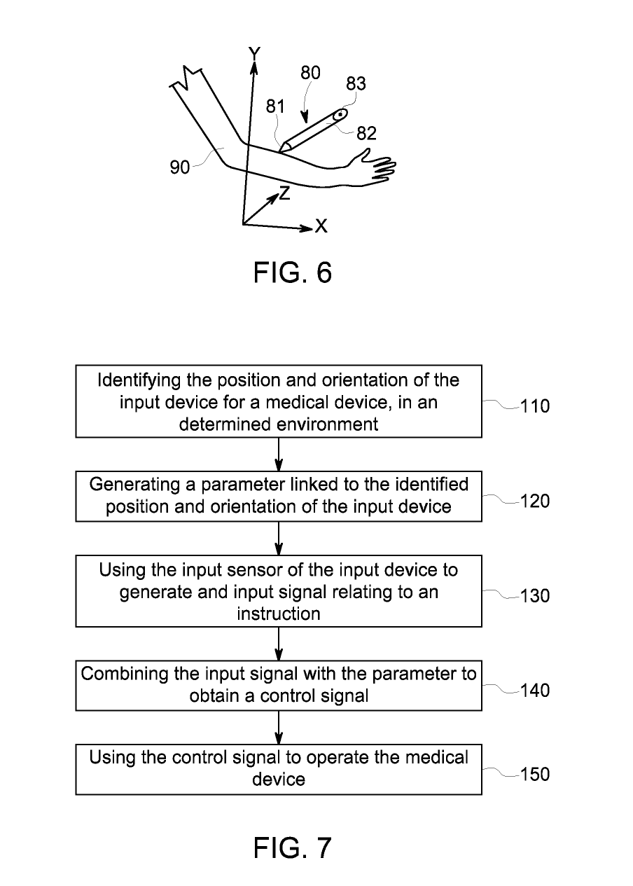

With reference to FIG. 6, a further embodiment of the invention is described. FIG. 6 shows an input device 80 which, in one embodiment, is a pointer. If an operator desires information about an area of interest, such as the arm 90 of a person 11, the input device 80 is directed to said area of interest. The input device 80 comprises a leading end 81 and a trailing end 82. The leading end 81 is configured to point to and indicate the specific area of interest. The trailing end 82 could be provided with an input sensor in the form of a button 83 to provide an input signal which is linked to an instruction for a medical device to which the input device 80 is connected.

The pointed device 80 in combination with at least one embodiment of the invention is particularly useful when an operator has obtained in a first step physiological data of an area of interest, such as the arm 90 of a person 11. The data relating to the arm 90 is, for example, obtained by radiology. In the context of this disclosure radiology makes reference to any known image acquisition modality such as, for example, X-ray radiography, ultrasound, computed tomography and magnetic resonance imaging (MRI).

A database of image data including data relating to a full 3D structure of a body part of interest can be generated in connection with any of the image acquisition modalities used. It is known that in some imaging procedures, after generating such a database an operator uses processing techniques, for example on a computer, to obtain specific slices, meaning images taken under a certain angle in order to try to obtain a most appropriate representation of the body part on a 2D screen. According to the present disclosure, after obtaining the data by, a radiology procedure, an operator could return to the actual body part 90 and indicate the exact axes of interest of which he would like to obtain the image. That means that the operator could position the input device 80 at a preferred position and could turn the input device 80 to a preferred angular position with respect to the body part 90 in order to obtain in an intuitive manner an image of the body 90 part which relates to the position and the angular orientation of the input device 80 with respect to the actual body part 90.

A further use of the method and system according to the present disclosure is that the location of an input device can be monitored in order to avoid losing the input device. For example, if the input device is removed from the determined environment and is no longer visible in the determined environment, an alarm could be generated. A further possibility is to use the method and system according to the present disclosure to indicate the location of a medical device in the determined environment. For example, if the operator is provided with a bracelet, the operator could position his hand in the vicinity of a medical device to locate the medical device in the determined environment. Thereafter, the actual position of the medical device in the determined environment could be obtained and/or compared with a preferred location for the medical device in the determined environment.

According to the present disclosure a single input device can be used to generate instructions for either a single medical device or several medical devices. In the determined environment, either one or several input devices are used. In case of multiple input devices, these can be used by either one or several operators. The observation sensor used to determine the location of the input device is either fixed to part of the determined environment or attached to the input device itself.

FIG. 7 shows a flow chart of an embodiment of the method of the present disclosure. According to FIG. 7 in a first step 110 the orientation, location, or both of the input device is identified in a determined environment.

In a second step 120 a parameter is generated linked to either or both of the identified orientation or location of the medical device and the identified orientation or location of the input device in the determined environment.

In a third step 130 the input sensor of the input device is used to generate an input signal relating to an instruction for the medical device.

In a fourth step 140 the input signal from the input device is used in combination with the parameters to obtain and use a control signal for the medical device.

This written description uses examples to disclose example embodiments of the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

What is claimed is:

1. A method for generating a control signal for operating a medical device using an input device located in a determined environment, wherein the input device comprises an input sensor configured to generate instructions for the medical device, the method comprising:
identifying the location of the input device in the determined environment;
generating a parameter linked to the identified location of the input device in the determined environment;
using the input sensor of the input device to generate an input signal relating to an instruction for the medical device;
using the input signal in combination with the parameter to obtain the control signal for the medical device; and
using the control signal to operate the medical device.

2. The method according to claim 1, wherein the method further comprises:
identifying the location of the medical device in the determined environment; and
identifying the location of the input device with respect to the medical device.

3. The method according to claim 1, wherein the method further comprises:
identifying the location of a person in the determined environment; and
identifying the location of the input device with respect to the person.

4. The method according to claim 2, wherein the method further comprises:
determining a threshold value relating to a maximum distance between the medical device and the input device;
comparing the identified location of the medical in the determined environment with the identified location medical device in the determined environment to obtain a value relating to the distance between the medical device and the input device;
comparing the value relating to the distance between the medical device and the input device with the threshold value relating to a maximum distance between the medical device and the input device; and
allowing the control signal to operate the medical device only if the distance between the input device and the medical device is equal or below the threshold value.

5. The method of claim 1, wherein the method further comprises installing in the determined environment at least one observation sensor, configured to observe the location of the input device.

6. The method according to claim 5, wherein the observation sensor is configured for 3D vision.

7. The method according to claim 5, wherein the observation sensor is configured for electro-magnetism localization.

8. The method according to claim 5, wherein the observation sensor is configured for radio frequency (RF) localization.

9. A system for generating a control signal for operating a medical device in a determined environment, the system comprising:
an input device connected to the medical device with an input sensor configured to generate an input signal relating to an instruction for the medical device;
at least one observation sensor configured to determine the location of the input device in the determined environment, wherein the at least one observation sensor is configured to generate a parameter relating the observed location of the input device in the determined environment; and
a processor connected to the input device and the at least one observation sensor, wherein the process is configured to receive and process the input signal generated by the input device and the parameter generated by the at least one observation sensor to obtain as a result of said processing the control signal for the medical device.

10. Method for generating a control signal for operating a medical device by an input device in a determined environment, wherein the input device is configured to generate instructions for said medical device, the method comprising:
identifying the orientation of the medical device in the determined environment;
identifying the orientation of the input device in the determined environment by an electronic compass;
using the identified orientation of the medical device and the identified orientation of the input device to identify a relative orientation of the input device with respect to the medical device;
generating a parameter linked to said relative orientation of the input device with respect to the medical device;
using the input device to generate an input signal relating to an instruction for the medical device; and
using the input signal and the parameter to obtain a control signal for the medical device.

11. Method according to claim 10, wherein the method comprises, after the identification of the position and the orientation of the medical device in the determined environment:
forwarding the identified orientation of the medical device in the determined environment to the input device; and
calibrating the electronic compass to match the identified orientation of the medical device.

12. The method according to claim 10, wherein the method further comprises:
identifying an updated orientation of the medical device in the determined environment;
identifying the orientation of the input device in the determined environment by an electronic compass; and
using the identified updated orientation of the medical device and the identified orientation of the input device to identify an updated relative orientation of the input device with respect to the medical device.

13. Method according to claim 12, wherein the method further comprises:
generating an updated parameter linked to said updated relative orientation of the input device with respect to the medical device;
using the input device to generate an input signal relating to an instruction for the medical device; and
using the input signal and the updated parameter to obtain an instruction for the medical device.

14. Method according to claim 10, wherein the method further comprises identifying the orientation of the medical device in the determined environment by an electronic compass.

15. Method according to claim 11, wherein the method further comprises forwarding the identified orientation of the medical device in the determined environment to the input device via CAN protocol.

16. System for generating a control signal for operating a medical device in a determined environment, the system comprising:
a medical device configured to receive instructions by an input device;
an input device connected to the medical device with an input sensor for generating an input signal relating to an instruction for the medical device, the input device comprising an electronic compass for identifying the orientation of the input device in the determined environment; and
a processor connected to the input device, the processer being configured to receive and process data relating the orientation of the medical device in the determined environment, data relating to the orientation of the input device in the determined environment obtained by the electronic compass, and an input signal generated by the input sensor to obtain as a result of said processing the control signal for the medical device.

17. An input device for a medical device, the input device configured to be used in a determined environment, the input device having an input sensor for generating an input signal relating to an instruction for the medical device, wherein the input device is provided with an electronic compass to identify the orientation of the input device in said determined environment.
